(19) United States
(12) Patent Application Publication
(10) Pub. No.: US 2026/0026754 A1
(43) Pub. Date: Jan. 29, 2026

(54) Patient Support Apparatus for an Imaging Apparatus

(71) Applicant: Siemens Healthineers AG, Forchheim (DE)

(72) Inventors: Wilfried Schnell, Forchheim (DE); Hans Weber, Erlangen (DE); Antje Lohfink, Erlangen (DE); Christopher Hable, Erlangen (DE); Stephan Biber, Erlangen (DE)

(73) Assignee: Siemens Healthineers AG, Forchheim (DE)

(21) Appl. No.: 19/282,011

(22) Filed: Jul. 28, 2025

(30) Foreign Application Priority Data
Jul. 29, 2024 (DE) 10 2024 207 099.8

(51) Int. Cl. A61B 5/00; A61B 5/055

(52) U.S. Cl. CPC A61B 5/704; A61B 5/055; A61B 5/702

(57) ABSTRACT

A patient support apparatus for an imaging apparatus, including: a patient couch and a cushioning element, wherein the cushioning element is arranged on a surface of the patient couch and forms a receiving surface for a patient, wherein the patient couch is designed to accommodate a patient in a recumbent posture, and wherein the patient support apparatus is designed to transport the patient on the patient couch into a patient receiving zone of the imaging apparatus, wherein a width of the cushioning element along an X direction of the patient support apparatus substantially corresponds to a width of the patient receiving zone at a receiving height for the patient couch.

### TECHNICAL FIELD

Independent of the grammatical term usage, individuals with male, female or other gender identities are included within the term.

The aspects of the disclosure relate to a patient support apparatus for an imaging apparatus.

### BACKGROUND

Comfortable support for and immobilization of patients represent advantageous preconditions for diagnostic imaging examinations. Not just the wellbeing of the patients is relevant in this case, but also a quick and error-free workflow in the preparation of the patients. In particular, by a suitable positioning of patients in a final position for the imaging examination, risks of jamming can be reduced, as also can image artifacts due to movement.

Conventional imaging apparatuses, such as e.g., magnetic resonance devices or computed tomography systems, typically comprise patient couches which are narrower than a patient receiving zone of the imaging apparatus. Padded lying surfaces of the patient couches in particular are generally narrower than the patient. When positioning the patient, care must be taken to ensure the patient does not collide with a wall of the patient receiving zone and maintains the necessary safety distance from the wall of the patient receiving zone.

The patient couches of current imaging apparatuses mostly feature thin and flat mattresses. The mattresses consist of a maximally homogeneous plastic layer which can be attached to spine coils, for example. Typically, the patient couch and the mattress are much narrower than an available width of the patient receiving zone since a single model of the patient couch is used for multiple models of the imaging apparatuses having patient receiving zones of different sizes. In particular, with imaging apparatuses having large patient receiving zones with a diameter of 70 cm and more, there is therefore the problem that anatomical regions at the sides of the patient (e.g., the arms and/or the legs) are guided by way of stationary parts of the patient support apparatus or rub against them when the patient couch is being moved.

For the reasons cited, imaging examinations require a painstaking preparation of the patient. This includes for example an individual positioning of support wedges, sandbags, retaining belts and positioning aids of various designs, which immobilize the patient in a predetermined pose. For example, the arms and/or other extremities of the patient are fixed by means of belt straps, which is time-consuming. Parts of the body of the patient that project beyond padded sections of the lying surface must be supported from underneath by means of dedicated pads. Furthermore, tubes and cables must be fixed in place such that they are located close to the patient and do not fall down or become jammed between the patient couch and the wall of the patient receiving zone when the patient couch is being moved. If parts of the body of the patient are resting on the lying surface outside of padded sections, this can be uncomfortable for the patient and can cause said parts of the body to rub against sections of the enclosure of the imaging apparatus when the table is being moved. The patient must then be slowly introduced into the patient receiving zone by means of the patient support apparatus in order to check whether there is sufficient clearance from a wall of the patient receiving zone.

### SUMMARY

It is an object of the aspects of the disclosure to facilitate a preparation of a patient for an imaging examination.

This object is achieved according to the aspects of the disclosure by means of the subject matter of the independent claims. Advantageous aspects and beneficial developments are the subject matter of the dependent claims.

The patient support apparatus according to the aspects of the disclosure is designed to accommodate a patient during an imaging examination by means of an imaging apparatus.

The patient support apparatus comprises a patient couch and a cushioning element. The cushioning element is arranged on a surface of the patient couch and forms a receiving surface for a patient.

A patient couch may have a table or a substantially planar surface. The patient couch is preferably designed to receive a patient in a predetermined examination position and to transport the patient by means of the patient support apparatus into a patient receiving zone, in particular an imaging region or image acquisition region, of the imaging apparatus. The predetermined examination position of the patient on the patient couch may comprise a predetermined or desired arrangement of the patient relative to the imaging apparatus during the imaging examination. In particular, the predetermined examination position of the patient on the patient couch may be characterized by a reclined posture or a partly reclined posture of the patient on the patient couch (e.g., a slight inclination of the upper body of the patient relative to the patient couch and/or one or more extremities laid or supported thereon).

According to the aspects of the disclosure, the patient support apparatus is designed to transport the patient on the cushioning element by means of the patient couch into a patient receiving zone of the imaging apparatus.

The patient support apparatus may comprise a motor and/or an actuator which are/is designed to position the patient couch variably relative to the patient support apparatus or a section of the patient support apparatus. It is conceivable that the motor and/or the actuator are designed to transport the patient by means of the patient couch variably along at least one spatial direction, preferably at least two spatial directions oriented perpendicularly to one another.

The cushioning element can be designed to cover a surface or a section of the patient couch, partly or completely. In particular, the cushioning element can be designed to be reversibly deformed upon receiving the patient. The cushioning element may, for example, comprise an elastic element, a reversibly deformable element, and/or a fillable or inflatable element that is designed to be molded to fit the shape of an anatomical region of a patient positioned on the patient couch.

The cushioning element may comprise an elastic material, in particular a reversibly deformable material, or consist of such a material. For example, the cushioning element may consist of a foam material. However, it is also conceivable that the cushioning element comprises one or more pockets or pouches. The pockets or pouches can be designed to be filled collectively or separately from one another with a fluid in order to allow an adjustment of an elasticity and/or a dimension of one more sections of the cushioning element.

The cushioning element is preferably arranged on a surface of the patient couch that points counter to a direction of the force of gravity.

The patient couch is preferably designed to receive a patient in a recumbent posture. It is conceivable that a longitudinal dimension of the patient couch corresponds at least to a longitudinal dimension of a patient. For example, the longitudinal dimension of the patient couch may amount to at least 2 m or exceed 2 m. A width of the patient couch may amount to at least 30 cm, at least 40 cm or at least 50 cm. The width of the patient couch may be understood as a cross-sectional dimension of the patient couch along an X direction of the patient support apparatus. The X direction of the patient support apparatus preferably coincides with an X direction of the imaging apparatus.

A width of the patient support apparatus may exceed the width of the patient couch. It is equally conceivable that a width of the cushioning element exceeds the width of the patient couch. The width of the cushioning element and the width of the patient support apparatus may be understood as cross-sectional dimensions along an X direction of the patient support apparatus. An X direction may be oriented parallel to a transverse plane of a patient positioned in an appropriate manner for examination on the patient couch. In particular, the X direction can be oriented parallel to a transverse plane and a frontal plane of a patient positioned in an appropriate manner for examination on the patient couch. An appropriate position of the patient on the patient couch may be characterized in that the patient is arranged in a recumbent and/or outstretched posture in the longitudinal direction on the cushioning element of the patient support apparatus.

According to the aspects of the disclosure, a width of the cushioning element along an X direction of the patient support apparatus substantially corresponds to a width of the patient receiving zone at a receiving height for the patient couch. The width of the cushioning element may deviate, for example, by less than 5 cm, less than 4 cm, or preferably less than 3 cm from the width of the patient receiving zone at a receiving height for the patient couch.

The receiving height for the patient couch may substantially coincide with a geodetic height of the cushioning element arranged on the patient couch. The receiving height for the patient couch may also be characterized by a distance of a main surface of the cushioning element from a floor area on which the patient support apparatus is positioned. In particular, the receiving height for the patient couch may correspond to a vertical distance between a surface of the patient couch facing toward the cushioning element and the floor area. Preferably, the receiving height for the patient couch corresponds to a height of the patient receiving zone at which the cushioning element is introduced into the imaging region of the imaging apparatus during the transportation of a patient by means of the patient support apparatus. The receiving height for the patient couch may also be understood as a height of the patient couch at which it is driven into an imaging region of an imaging apparatus. It is conceivable that the receiving height for the patient couch coincides with the geodetic height of the cushioning element arranged on the patient couch with a deviation of less than 5 cm, less than 4 cm, or less than 3 cm.

In an aspect, a width of the patient couch along an X direction of the patient support apparatus is less than the width of the patient receiving zone at the receiving height for the patient couch. In this case the width of the cushioning element may exceed the width of the patient couch. In particular, the cushioning element may project or protrude beyond at least one longitudinal edge, preferably two longitudinal edges, of the patient couch. The patient support apparatus may comprise sections, such as e.g., braces or struts, which are designed to support or carry the sections of the cushioning element that protrude beyond the patient couch.

A patient support apparatus comprising a cushioning element according to the aspects of the disclosure can cover a surface of the patient couch facing toward the patient and/or of the patient support apparatus completely or on selected sections. This advantageously enables a contact and/or a friction between anatomical regions of a patient positioned in an appropriate manner for examination on the patient couch and stationary sections of the patient couch and/or of the patient support apparatus to be avoided during a transportation of the patient by means of the patient support apparatus.

In addition, a patient support apparatus comprising a cushioning element according to the aspects of the disclosure can underpad or cushion the body of the patient over the entire surface or on selected body parts. Patient comfort can advantageously be increased as a result and a risk of an imaging examination being prematurely terminated by the patient can be reduced. In particular, extremities of the patient can be arranged over an entire available width of the patient support apparatus and/or the patient couch without the risk of the patient coming into contact with stationary and/or unpadded sections of the patient support apparatus and/or of the patient couch. This advantageously enables a process involving a preparation, in particular a positioning, of patients for an imaging examination to be simplified or shortened.

Furthermore, a cushioning element according to the aspects of the disclosure can be adjusted to conform to the body of the patient or snugly fit the same. This enables a smaller distance to be realized between a diagnostically relevant body region and a local coil integrated into the patient couch (such as, e.g., a spine coil), as a result of which a signal-to-noise ratio of the local coil can advantageously be increased.

In particular, the cushioning element can cover an entire lying area facing away from the imaging apparatus in order to facilitate a simple, quick, and comfortable support for the patient in a “feet first” position. Currently, the majority of magnetic resonance examinations are performed using “head first” positioning. This is very unpleasant in particular for claustrophobic patients. A positioning in the “feet first” position, on the other hand, is awkward and often uncomfortable for patients due to a lack of padding. As a result of a lack of underpadding, patients become restless more quickly, and this can lead to a degradation of image quality due to increased movement. In order to conduct examinations in a “feet first” position and “head first” position easily, comfortably, without complications and quickly, a cushioning element which covers an entire table area of the patient support apparatus and at the same time closely conforms to the body of the patient and provides support for the patient is of great advantage.

In an aspect, the patient support apparatus according to the aspects of the disclosure comprises a side element which is arranged along a longitudinal edge of the patient couch. The side element is designed to limit or enclose a patient positioned in an appropriate manner for examination on the patient couch on one side, along at least a part of a longitudinal extension of the patient.

The side element can be designed to prevent a movement of a part of the body of a patient positioned in an appropriate manner for examination on the patient couch beyond a receiving surface for the patient defined by the cushioning element and/or the patient couch. In particular, the side element can be designed to prevent or avoid a contact between a part of the body of the patient positioned in an appropriate manner for examination on the patient couch and a wall of the patient receiving zone of the imaging apparatus and/or a stationary section of the patient support apparatus.

For example, the side element can be embodied as a slat, a bar, a strip, a rail, a barrier, or the like. The side element may have a planar shape with a substantially rectangular cross-section. However, it is also conceivable that the side element has a complex geometric shape. For example, the side element may have a C-shaped, a V-shaped, or a U-shaped cross-section in the axial direction. In particular, a shape of an axial cross-section may be matched to or contoured to fit a curvature of a wall of the imaging region of the imaging apparatus. This advantageously allows the side element to be arranged in a particularly space-saving manner on the patient couch.

In a preferred aspect, the side element projects partly or completely beyond a surface provided by the patient couch and/or the cushioning element for receiving the patient.

The side element can be mechanically connected to the patient couch, the cushioning element, and/or the patient support apparatus. In a preferred aspect, the side element is mechanically connected to the patient couch and/or the patient support apparatus. It is conceivable that the side element is connected to the patient couch and/or the patient support apparatus by means of an arm, a strut, or an arbitrary retainer. A mechanical connection for securing the side element and the cushioning element, the patient couch, and/or the patient support apparatus may comprise a force-fit connection, a form-fit connection, and/or a material-to-material bond. Possible examples of such connections include a screwed connection, a clamped connection, a latching mechanism, a crimped connection, a snap-fit connection, an adhesive bond, a welded joint (e.g., a friction welded joint), or the like.

A longitudinal edge of the patient couch can constitute an edge of the patient couch that is aligned along or parallel to a longitudinal extension of the patient couch. The longitudinal edge of the patient couch can constitute an edge having the greatest dimension or main dimension of the patient couch.

In a preferred aspect, the patient support apparatus has a first side element and a second side element which are arranged on opposite longitudinal edges of the patient couch and are designed to limit or flank a patient positioned in an appropriate manner for examination on the patient couch on two opposing sides along at least a part of a longitudinal extension of the patient. The first side element and the second side element can be implemented according to an above-described aspect of a side element.

By means of a patient support apparatus having a side element according to the aspects of the disclosure, a contact of a patient positioned in an appropriate manner for examination on the patient couch with a component of the patient support apparatus and/or of the imaging apparatus can advantageously be avoided when an imaging examination is performed. In particular, extremities of the patient, but also tubes, cables, local coils, or other components or instruments required for performing the imaging examination, are secured against slipping off from the patient couch and/or becoming jammed between the patient couch and a wall of the imaging apparatus. The side elements may, for example, form a trough in which cables and tubes are laid loose and at the same time are secured such that additional safeguarding steps are advantageously unnecessary.

In an aspect, the side element has a padding on a surface directed toward the patient couch. A padding on an inside of the side element ensures the patient is comfortably supported and enables additional cushioning steps to be dispensed with. In an aspect of the patient support apparatus as a mobile patient table, the side elements can advantageously be used as a safeguard for the patient during transportation.

In a preferred aspect, the patient support apparatus according to the aspects of the disclosure comprises a joint.

A joint may be embodied as any rotatable or movable connecting element. For example, the joint may comprise a flexible element, a rotary bearing, a hinge, a guiding element, an angle joint, a ball joint, a radial bearing, an axial bearing, a linear guide, an axle, and/or a shaft.

In an aspect, the side element is mechanically connected by means of the joint to the patient support apparatus and/or the patient couch. For example, a first section of the joint can be mechanically connected to the patient couch and/or the patient support apparatus, while a second section of the joint is mechanically connected to the side element. The joint can be designed to change a spatial position and/or alignment of the first section relative to the second section continuously or discontinuously. In particular, the joint can be designed to guide the first section along a predetermined movement trajectory relative to the second section.

The joint is designed to variably change a spatial arrangement of the side element relative to the patient couch.

For example, the joint may comprise an axial bearing which is designed to enable the side element to rotate about an axis of rotation which is arranged substantially parallel to the longitudinal edge of the patient couch. In this case, an orientation of the axis of rotation of the axial bearing may deviate by less than 10°, less than 8°, less than 6°, or preferably less than 4° from an orientation of the longitudinal edge of the patient couch.

In a further example, the joint comprises a flexible element which is designed to enable the side element to be folded or folded in. In particular, the flexible element can define a folding edge along which the side element is pivotably mounted. The flexible element may be embodied for example as a folding joint or a folding element. The flexible element preferably consists of a flexible or elastic material, such as e.g., a natural or synthetic fabric, an elastic foam material and/or an elastomer.

A patient support apparatus comprising a joint according to the aspects of the disclosure permits the side element to be folded in over a patient positioned in an appropriate position for examination on the patient couch. This allows extremities of the patient covered by the side element to be immobilized in a predetermined position, as a result of which movements of the patient during an imaging examination and motion artifacts associated therewith can advantageously be reduced or prevented.

In a further aspect, the patient support apparatus according to the aspects of the disclosure has an actuator which is designed to guide the side element variably by means of the joint along a movement trajectory defined by the joint.

An actuator can be any arbitrary drive-oriented unit which is designed to convert a control signal into a mechanical movement of the joint and/or of the side element. For example, the actuator can be embodied as an electric motor, a bimetallic actuator, a hydraulic actuator, a pneumatic actuator, an electrochemical actuator, an electromechanical actuator, a piezoactuator, a magnetostrictive actuator, a shape memory alloy actuator, an electroactive polymer actuator or the like.

In an example, the side element is embodied as an inflatable side wing. The actuator can be implemented correspondingly as a hydraulic or pneumatic actuator, which is designed to fill the side wing with a hydraulic or pneumatic fluid (e.g., a compatible liquid or an inert gas). In a further example, the side element is embodied as a rigid or semi-rigid side wing. In this case, the actuator can be implemented as a piezoactuator or an electric motor, which is designed to move the joint and/or the side element mechanically.

In a preferred aspect, the actuator is designed to be compatible with a magnetic resonance device. This can mean that magnetic components and/or electrical conductors of the actuator are integrated in a carrier structure of the patient support apparatus. The patient support apparatus can have a shield that reduces or prevents unwanted interactions between magnetic components and/or electrical conductors and a magnetic field of the magnetic resonance device. It is further conceivable that the actuator avoids as far as possible components and/or materials which may interact in an undesirable manner with the magnetic field of the magnetic resonance device.

The actuator can be embodied to move the side element along the movement trajectory defined by the joint as a function of a control signal of a control unit. In particular, the actuator can be embodied to transfer the side element automatically from an open position into an examination position (and vice versa). The examination position may be characterized in that a patient positioned in an appropriate manner for examination on the patient couch is limited or encompassed at the sides by one or more side elements. The open position may be characterized by an arrangement of the side elements that allows a patient to access the patient couch and/or the cushioning element from at least one side. For example, in an open position, the side elements can be arranged at the side of the patient support apparatus beneath a plane defined by the patient couch.

The control unit can be embodied as an autonomous control unit, a control unit of the patient support apparatus, and/or a control unit of the imaging apparatus.

A patient support apparatus having an actuator according to the aspects of the disclosure can enable an automatic transition of the side element from an open position into an examination position. An access by the patient to the patient couch, but also a positioning of the patient on the patient couch, can advantageously be simplified as a result. Furthermore, an awkward manual positioning of the side element by a user can be avoided, thereby advantageously increasing an efficiency of a preparation of the patient for an imaging examination.

According to an aspect, the side element of the patient support apparatus according to the aspects of the disclosure has a flexible section which is designed to be adjusted to conform to a surface of a patient positioned in an appropriate manner for examination on the patient couch.

The flexible section of the side element may correspond to a flexible element according to an above-described aspect. It is conceivable that the side element comprises a plurality of flexible elements, in particular, a plurality of folding joints.

It is conceivable that the flexible section comprises a reversibly deformable material or consists of such a material. For example, the flexible section may consist of a reversibly deformable material which allows the flexible section to be adjusted to conform to a shape and/or a body part of a patient. The reversibly deformable material is preferably deformable by a manual application of force by a user and/or by the patient. Examples of reversibly deformable materials include fabrics made of natural fibers or cotton, but also synthetic materials, such as e.g., thermoplastic polymers, elastomers, foam materials, and the like.

A patient support apparatus according to the aspects of the disclosure enables the side element to be adjusted to conform in shape to an anatomical region of a patient positioned in an appropriate manner for examination on the patient couch. An immobilization of the patient during an imaging examination can be further improved as a result, thereby enabling image artifacts occurring due to movements of the patient to be reduced or prevented.

In a preferred aspect, the patient support apparatus according to the aspects of the disclosure comprises a fixing element which is designed to secure the side element in a predetermined arrangement relative to the patient couch and/or to a patient positioned in an appropriate manner for examination on the patient couch.

The fixing element can be designed to secure the side element in an examination position. The examination position may be characterized in that the side element bears against or rests on a body part of the patient and/or is adjusted to conform to the body part.

The fixing element may comprise any desired fastening mechanism that is suitable for securing the side element in the examination position. It is conceivable that the fastening mechanism comprises two complementary parts that are designed to interengage and provide a mechanical connection. For example, a first part of the fastening mechanism can be arranged on the side element, while a second part of the fastening mechanism is arranged on the patient couch, the cushioning element, and/or the patient support apparatus. It is conceivable that the fixing element at least partly encompasses the patient. The fixing element may be further embodied to be connectable to two opposing side elements or opposing sides of the patient couch and/or the patient support apparatus.

For example, the fixing element may have a hook and a loop. The hook can be designed to engage in the loop in order to lock the side element in the examination position. Similarly, the fixing element may comprise a belt and a buckle, a hook-and-loop fastener, or positive-locking clips. It goes without saying that further fastening mechanisms that allow a reversible securing of the side element in the examination position are conceivable.

A patient support apparatus having a fixing element according to the aspects of the disclosure advantageously enables an improved immobilization of a body part of the patient covered by the side element without the need to cover the patient completely or over the entire surface. This advantageously enables a tolerance of the patient toward the immobilization to be increased.

In an aspect of the patient support apparatus according to the aspects of the disclosure, the cushioning element and/or the patient couch have a recess which is designed to accommodate a component of the imaging apparatus.

A component of the imaging apparatus may constitute, for example, a hose, a cable, or a part of a local coil of the imaging apparatus.

The recess can assume any position on the cushioning element. Preferably, the recess is arranged at an edge or a peripheral region of the cushioning element such that a width of the cushioning element is reduced compared to a section without recess.

Components of the imaging apparatus can be guided through the recess and/or arranged in the recess. This advantageously enables a detrimental impact on a patient due to components of the imaging apparatus, but also a snagging of the components between the patient support apparatus and a wall of the imaging apparatus, to be avoided.

In an aspect, the cushioning element has a first section and a second section, a width of the second section being reduced compared to a width of the first section.

The cushioning element may have a plurality of sections. Preferably, two or more sections of the plurality of sections are mechanically connected to one another. For example, the sections of the cushioning element may be aligned in a row or be arranged adjacent to one another.

A first section of the cushioning element may have a substantially rectangular base area. A second section of the cushioning element may have a substantially trapezoidal or conical base area. In particular, the second section may become narrower or steadily reduce in width in the X direction along a Z direction or a longitudinal extension of the patient couch. It is further conceivable that the second section has a substantially rectangular basic shape, which has a reduced width compared to the first section. Furthermore, the width of the cushioning element along a Z direction, in particular the width of the first section and the second section, may correspond to a width of the patient couch and/or of the patient support apparatus along the Z direction or be different therefrom.

According to an above-described aspect, the second section may be designed to provide one or more recesses for a component of the imaging apparatus. In particular, the recesses in the second section may be designed to allow a local coil to slide along a surface of the patient couch. For example, the recesses can reduce a width of the cushioning element in such a way that the local coil on the patient couch can slide past the second section of the cushioning element or borders or encloses the second section of the cushioning element on two sides.

A patient support apparatus having a cushioning element with a recess advantageously permits components of an imaging apparatus to be guided through and/or housed. In particular, a cushioning element according to the aspects of the disclosure enables a local coil to be shifted along a longitudinal direction of the patient support apparatus. This advantageously enables a technical overhead which is associated with a guidance system for a local coil integrated into the patient couch to be reduced.

In an aspect of the patient support apparatus according to the aspects of the disclosure, the width of the cushioning element along an entire longitudinal extension of the patient couch substantially coincides with the width of the patient receiving zone at the receiving height for the patient couch.

At the receiving height for the patient couch, the width of the cushioning element may deviate by a few millimeters from the width of the patient receiving zone. Preferably, the width of the cushioning element is substantially constant along the longitudinal extension of the patient couch. This may mean that the width of the cushioning element varies by a few millimeters along the longitudinal extension of the patient couch.

It is conceivable that the cushioning element has one or more recesses that are arranged in the cushioning element at a distance from an edge of the cushioning element. The cushioning element may therefore have recesses for accommodating a component of an imaging apparatus without the cushioning element having a section with a reduced width along the longitudinal extension of the patient couch.

A patient support apparatus having a cushioning element according to the aspects of the disclosure advantageously enables the available receiving surface for the positioning of the patient on the cushioning element to be maximized.

According to a further aspect, the cushioning element of the patient support apparatus according to the aspects of the disclosure has a plurality of elastic elements. The elastic elements are designed to support selected anatomical regions of a patient when the patient is positioned in an appropriate manner for examination on the patient couch.

A first elastic element and a second elastic element of the plurality of elements may be separate or demarcated from one another. It is also conceivable that a first elastic element and a second elastic element of the plurality of elastic elements, when arranged in an appropriate manner for use on the patient couch, protrude at different heights from a side of the patient couch facing toward the cushioning element. For example, the first elastic element and the second elastic element can have different dimensions, in particular, different heights.

The patient support apparatus may further comprise a third elastic element and/or a fourth elastic element. One or more dimensions of the third elastic element and/or of the fourth elastic element may be different from one or more dimensions of the first elastic element and/or of the second elastic element.

A thickness or height of an elastic element can amount to approx. 3 cm. It is, however, conceivable that one or more elastic elements of the plurality of elastic elements are embodied thinner or thicker, in particular, thicker by a multiple.

An elastic element may comprise an elastic material or consist of such a material. For example, an elastic element may comprise a foam material, a fibrous material, a rubber, or one or more spring elements (e.g., coil innersprings). In particular, the elastic element may be embodied as a cushion or pouch filled with a foam material or fibrous material. However, it is also conceivable that the elastic element is embodied as fillable with a hydraulic or pneumatic fluid in order to enable a dimension and/or an elasticity of the elastic element to be adjusted. The elastic element preferably consists of a plastic material, in particular a synthetic elastomer, in order to ensure that the elastic element is impermeable to hydraulic or pneumatic fluids.

The elastic elements of the plurality of elastic elements of the cushioning element are preferably mechanically connected or coupled to one another. For example, the elastic elements can be arranged in a predetermined matrix (e.g., in a lozenge pattern, a rectangle pattern or a triangle pattern). It is also conceivable that the elastic elements are arranged contiguously to one another. The elastic elements can be stitched, welded or adhesively bonded to one another. In particular, the elastic elements can be arranged using a discrete quilting technique. In addition, the elastic elements may consist of one or more molded elements which are connected to one another by means of a material bond.

The comfort of a patient positioned in an appropriate manner for examination on the patient couch can advantageously be increased by means of a patient support apparatus having a cushioning element according to the aspects of the disclosure. As a result, a risk of an imaging examination being terminated prematurely can advantageously be reduced and/or a duration of an imaging examination tolerable for the patient increased.

In a further aspect of the patient support apparatus according to the aspects of the disclosure, a first elastic element and a second elastic element of the plurality of elastic elements project or protrude at different heights from a side of the patient couch facing toward the cushioning element when the cushioning element is arranged in an appropriate manner for use on the patient couch.

It is conceivable that a surface of a first elastic element of the plurality of elastic elements facing away from the patient couch protrudes from a surface of a second elastic element of the plurality of elastic elements facing away from the patient couch. For example, a height or a thickness of the first elastic and of the second elastic element may be different.

The cushioning element and the plurality of elastic elements may be designed according to an above-described aspect.

A patient support apparatus having an elastic element according to the aspects of the disclosure advantageously enables a tailored support for dedicated anatomical regions of a patient positioned in an appropriate manner for examination on the patient couch. In particular, one or more elastic elements having different thicknesses or heights can enable extremities or other anatomical regions of the patient to be arranged at different distances from the patient couch and/or in postures required for specific imaging examinations.

In a further aspect, the patient support apparatus according to the aspects of the disclosure comprises a shaping device which is designed to adjust an elasticity and/or a dimension of an elastic element of the plurality of elastic elements in accordance with an imaging examination.

For example, the shaping device may comprise an electroactive polymer, an electromechanical converter, a bimetallic actuator, and/or an actuator according to an above-described aspect. The shaping device can be designed to set or adjust the elasticity and/or the dimension of one or more elastic elements of the plurality of elastic elements as a function of a control signal. The shaping device is preferably connected to a signal connection of a control unit. The control unit can be designed to transmit a control signal to the shaping device by means of the signal connection and to direct the shaping device to adjust the elasticity and/or dimension of the one or more elastic elements.

In a preferred aspect, the control unit is designed as a control unit of the imaging apparatus or is connected to the control unit of the imaging apparatus by means of a signal connection. The control unit can be designed to adjust the elasticity and/or dimension of one or more elastic elements of the plurality of elastic elements by means of an actuation of the shaping device. The control unit can in particular be designed to adjust an elasticity and/or a dimension of one or more elastic elements by means of an actuation of the shaping device in such a way that an anatomical region and/or an extremity of a patient is stabilized and/or supported in a desired posture and/or at a desired spatial position in accordance with an imaging examination that is to be performed.

A patient support apparatus having a shaping device according to the aspects of the disclosure can improve or facilitate a positioning and/or support of an anatomical region of a patient in accordance with an imaging examination that is to be performed. In particular, the patient support apparatus according to the aspects of the disclosure can enable an automated positioning and/or support of an anatomical region of a patient in accordance with an imaging examination that is to be performed. A process for a positioning and/or preparation of an imaging examination of a patient can advantageously be simplified and/or completed faster as a result.

In an aspect of the patient support apparatus according to the aspects of the disclosure, the plurality of elastic elements comprise at least one fillable element. The shaping unit is designed to fill the at least one fillable element with a fluid in order to change the elasticity and/or the dimension of the at least one element.

The fillable element can constitute any desired fluid-tight container having a flexible and/or elastic wall. The fillable element preferably has an opening, a nozzle, or a connector that is designed to connect a volume encompassed by the fillable element to a fluid line. It is conceivable that the fillable element is connected to the shaping device and/or to a fluid reservoir by means of the fluid line. The shaping device, the fluid line, and/or the fluid reservoir preferably comprise a conveyor unit which is designed to transport a pneumatic or hydraulic fluid into the fillable element. In particular, the shaping device can be designed to actuate the conveyor unit in order to transport the pneumatic or hydraulic fluid into the fillable element. The shaping unit may be understood as a pneumatic or hydraulic actuator according to an above-described aspect.

The fluid may comprise a gaseous medium or a liquid medium. In particular, the fluid may comprise a pneumatic fluid, such as e.g., air or an inert gas, or a hydraulic fluid, such as e.g., oil or water.

By using a cushioning element comprising a fillable element, it is advantageously possible to avoid electrical drive components for adjusting the elasticity and/or dimension of an elastic element (or electrical conductors required for this purpose) in an imaging region of an imaging apparatus. In particular, unwanted interactions of the electrical drive components with an imaging principle of the imaging apparatus, such as e.g., interactions with a magnetic field of a magnetic resonance device, can advantageously be reduced or avoided.

The imaging apparatus according to the aspects of the disclosure comprises an image acquisition region and a patient support apparatus according to an above-described aspect.

An imaging apparatus can be any device that is designed to acquire image data of an object, in particular of a patient or of a part of the body of a patient. Preferably, the imaging apparatus is designed to acquire two-dimensional and/or three-dimensional image data, in particular time-dependent three-dimensional image data, of the object. The imaging apparatus is designed to acquire medical or diagnostic image data of an object positioned in the image acquisition region. Examples of imaging apparatuses include magnetic resonance devices, X-ray machines, computed tomography devices, single-photon emission computed tomography systems, positron emission tomography systems, but also mammography devices. In a preferred aspect, the imaging apparatus is implemented as a magnetic resonance device.

An image acquisition region can constitute a section of the imaging apparatus that is designed to accommodate the object during an imaging examination. In particular, the image acquisition region may comprise an imaging volume (or field of view) from which image data is acquired during the imaging examination.

Preferably, the imaging apparatus is designed to perform an imaging examination of an object that is positioned inside the image acquisition region of the imaging apparatus. The imaging apparatus can be designed, for example, to acquire magnetic resonance data, X-ray data, or computed tomography data of the object that is positioned inside the image acquisition region.

In a preferred aspect, the imaging apparatus is designed as a magnetic resonance device. The magnetic resonance device can be designed to acquire magnetic resonance image data, in particular diagnostic magnetic resonance image data, of the object positioned inside the image acquisition region. The object may be a human or animal patient, for example.

In a preferred aspect, the magnetic resonance device is designed as a closed scanner (“closed-bore scanner”) or as a scanner having a cylinder-shaped patient receiving zone or patient tunnel. A closed scanner can have a substantially cylindrically shaped image acquisition region. A main magnet of the closed scanner may comprise one or more magnetic coils which encompass the image acquisition region along an axial direction or an axis of rotation, in particular an axis of rotational symmetry, of the main magnet. A magnetic coil may comprise an electrical conductor having negligible electrical resistance at (or below) a superconducting temperature. A direction of a main magnetic field which is provided by means of the main magnet may be oriented substantially parallel to an access direction to the image acquisition region and/or the axial direction of the cylindrical bore.

It is also conceivable that the magnetic resonance device is designed as an open scanner (“open-bore scanner”). An open scanner may comprise two magnets that are arranged opposite each other, separated by the image acquisition region. A direction of the main magnetic field of the open scanner may be oriented substantially orthogonally to an access direction to the image acquisition region and/or the axial direction of the cylindrical bore.

The imaging apparatus according to the aspects of the disclosure shares the advantages of the patient support apparatus according to the aspects of the disclosure according to an above-described aspect. In particular, a process entailing a preparation and/or positioning of a patient for an imaging examination by means of a patient support apparatus according to the aspects of the disclosure can be simplified and/or completed faster.

In an aspect, the imaging apparatus according to the aspects of the disclosure has a control unit which is designed to actuate the shaping device to adjust an elasticity and/or a dimension of an elastic element of the plurality of elastic elements in accordance with an imaging examination.

As described above, the cushioning element of the patient support apparatus can comprise a plurality of elastic elements that are designed to support different anatomical regions of a patient when the patient is positioned in an appropriate manner for examination on the patient couch. The control unit can be designed to adjust an elasticity and/or a dimension of an elastic element of the plurality of elastic elements by means of an actuation of the shaping device in accordance with an imaging examination, in particular a magnetic resonance examination.

In a preferred aspect, the control unit is connected to the shaping device by means of a signal connection. The control unit can be designed to output a control signal that actuates the shaping device in order to adjust an elasticity and/or a dimension of an elastic element of the plurality of elastic elements. Preferably, the control unit and/or a computing unit of the imaging apparatus are/is designed to identify or register an imaging examination that is to be performed. In particular, the control unit can be designed to adjust an elasticity and/or a dimension of an elastic element of the plurality of elastic elements automatically or partly automatically by means of the shaping device in accordance with the imaging examination that is to be performed.

It is conceivable that the control unit is designed to register the imaging examination that is to be performed as a function of an input by a user, patient information, a (digital) patient record, and/or an entry in a radiological information system (RIS) or a hospital information system (HIS). For this purpose, the control unit can be connected to a memory unit, a network, in particular a local hospital network, and/or a cloud.

An imaging apparatus having a control unit according to the aspects of the disclosure can determine or register an imaging examination that is to be performed and adjust a characteristic of an elastic element of the plurality of elastic elements automatically by means of the shaping device in accordance with the imaging examination. Time and effort expended for preparing and/or performing an imaging examination can advantageously be reduced as a result.

In a preferred aspect, the imaging apparatus is designed as a magnetic resonance device. The cushioning element has a marker which represents a course of a projection of an imaging volume provided by the magnetic resonance device onto the cushioning element when the patient couch is transported into the patient receiving zone by means of the patient support apparatus.

The marker preferably represents a relative movement trajectory of the imaging volume provided by the magnetic resonance device toward the patient couch when the patient couch is transported into the patient receiving zone by means of the patient support apparatus. In particular, the marker can be designed to indicate to a user and/or a patient a position of the imaging volume relative to a surface of the cushioning element when the patient couch is driven into the patient receiving zone.

The marker may comprise a line, a dashed line, an icon, a pictogram, an image, a digit, a number, a letter, a word, or the like.

A marker according to the aspects of the disclosure permits information of a user and/or a patient about a spatial arrangement of the imaging volume of the magnetic resonance device relative to the patient couch. This enables a positioning aid to be provided which advantageously facilitates a centering or a left-to-right positioning of a patient on the patient support apparatus.

### BRIEF DESCRIPTION OF THE DRAWINGS

Further advantages and specific details will become apparent from the following description of exemplary aspects in conjunction with the schematic drawings, in which:

FIG. 1 shows an aspect of an imaging apparatus according to the aspects of the disclosure,

FIG. 2 shows an aspect of a patient support apparatus according to the aspects of the disclosure,

FIG. 3 shows an aspect of a patient support apparatus according to the aspects of the disclosure,

FIG. 4 shows an aspect of a patient support apparatus according to the aspects of the disclosure,

FIG. 5 shows an aspect of a patient support apparatus according to the aspects of the disclosure,

FIGS. 6a and 6b show an aspect of a patient support apparatus according to the aspects of the disclosure, and

FIG. 7 shows an aspect of an imaging apparatus according to the aspects of the disclosure.

### DETAILED DESCRIPTION

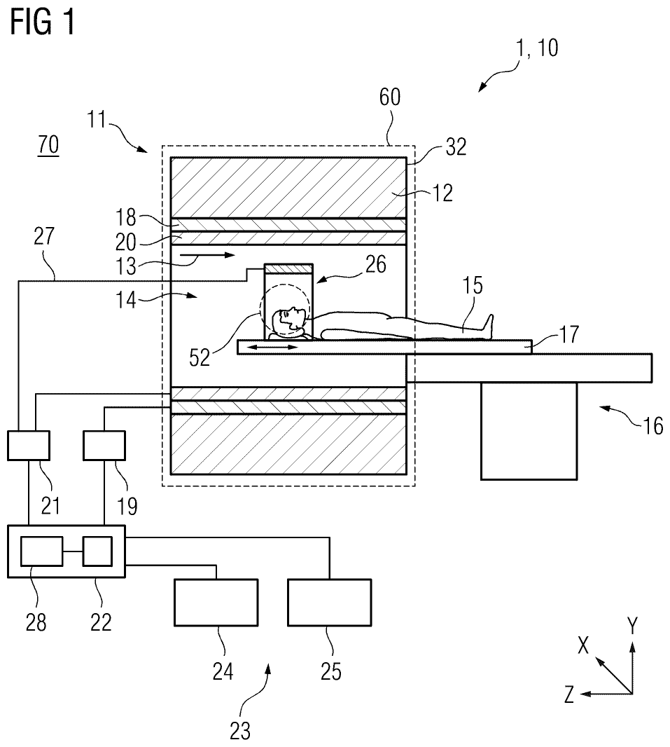

FIG. 1 shows an imaging apparatus 1 according to the aspects of the disclosure. In the example shown, the imaging apparatus 1 is embodied as a magnetic resonance device 10. The magnetic resonance device 10 comprises a field generation unit 11, which has a main magnet 12 comprising one or more permanent magnets, electromagnets, or superconducting magnets for generating a strong and in particular homogeneous main magnetic field 13 (B0 magnetic field). In addition, the magnetic resonance device 10 comprises a patient receiving zone 14, which is designed to accommodate an object or a patient 15 during a magnetic resonance examination. In the exemplary aspect shown, the patient receiving zone 14 is designed in a cylinder shape and is enclosed by the main magnet 12 along a circumferential direction. In principle, however, aspects of the patient receiving zone 14 differing from this example are also conceivable. The patient receiving zone 14 can substantially coincide with an imaging region or image acquisition region of the magnetic resonance device 10.

In the example shown in FIG. 1, the examination object is a patient 15. The patient 15 can be positioned in the patient receiving zone 14 by means of a patient support apparatus 16 according to the aspects of the disclosure. For this purpose, the patient support apparatus 16 comprises a patient couch 17, which is designed to be movable within the patient receiving zone 14.

The field generation unit 11 additionally comprises a gradient system, which is used for spatial encoding during a magnetic resonance examination. The gradient system may comprise one or more gradient coils 18 for generating magnetic gradient fields. The gradient coils 18 are driven by means of a gradient control unit 19 of the magnetic resonance device 10.

The field generation unit 11 further comprises a radiofrequency system having a radiofrequency antenna, which in the present exemplary aspect is implemented as a bodycoil 20 permanently integrated into the magnetic resonance device 10. The bodycoil 20 is designed to excite nuclear spins, which are located in the main magnetic field 13 generated by the main magnet 12. For this purpose, the bodycoil 20 is driven by a radiofrequency unit 21 of the magnetic resonance device 10 and radiates radiofrequency excitation pulses into the imaging region, which is substantially formed by the patient receiving zone 14 of the magnetic resonance device 10. The bodycoil 20 can be further designed to receive magnetic resonance signals from the imaging volume 52. The bodycoil 20 preferably constitutes a receive unit or a part of a receive unit of the magnetic resonance device 10.

The magnetic resonance device 10 may comprise further components, such as, e.g., a local coil 26. The local coil 26 can be placed in an appropriate position for use on a diagnostically relevant body region of the patient 15. The local coil 26 preferably comprises a plurality of antenna elements which are designed to capture magnetic resonance signals of the diagnostically relevant body region of the patient 15 and transmit them to the computing unit 28 and/or the control unit 22. For this purpose, the local coil can be connected to the radiofrequency unit 21 and the control unit 22 by means of an electric connecting cable 27. Analogously to the bodycoil 20, the local coil 26 can also be designed for exciting nuclear spins in the diagnostically relevant body region of the patient 15. The local coil 26 can be driven by the radiofrequency unit 21 for this purpose.

The magnetic resonance device 10 comprises a control unit 22 for controlling the magnetic resonance device 10, in particular the gradient control unit 19 and the radiofrequency unit 21. The control unit 22 is designed to control an execution of an imaging sequence, such as e.g., a GRE (gradient echo) sequence, a TSE (turbo spin echo) sequence or a UTE (ultra-short echo time) sequence. In addition, the control unit 22 comprises a computing unit 28 for evaluating magnetic resonance signals captured by means of an imaging sequence during a magnetic resonance examination.

The magnetic resonance device 10 may comprise a user interface 23, which has a signal connection to the control unit 22. Control information, such as e.g., imaging parameters of the magnetic resonance examination, can be displayed on a display unit 24 of the user interface 23. The display unit 24 may, for example, comprise a screen or monitor which is designed to provide a graphical user interface presenting imaging parameters of an imaging sequence and/or image data of a diagnostically relevant body region of the patient 15. The user interface 23 further comprises an input unit 25 by means of which parameters of a magnetic resonance examination can be input or modified by a user.

The magnetic resonance device 10 according to the aspects of the disclosure may, of course, comprise further components which are typical for magnetic resonance devices. The general mode of operation of a magnetic resonance device is well known to the person skilled in the art. A detailed description of further components or of a measurement data acquisition of a magnetic resonance examination is therefore dispensed with.

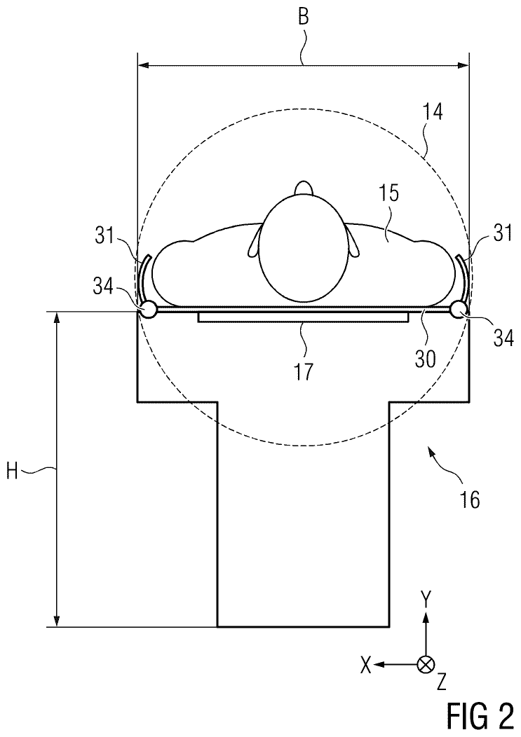

FIG. 2 shows an aspect of a patient support apparatus 16 according to the aspects of the disclosure. In the example shown, a patient 15 is placed in an appropriate position for examination on the cushioning element 30 of the patient support apparatus 16. A width of the patient couch 17 is in this case less than a width B of the cushioning element 30, which protects the patient 15 against contact with components of the patient support apparatus 16. The width B of the cushioning element 30 substantially corresponds to a width of the patient receiving zone 14 in the region of a receiving height H for the patient couch 17.

In the present example, the patient support apparatus 16 has two side elements 31 which limit or flank the patient 15 positioned in an appropriate manner for examination on the patient support apparatus 16 on two opposing sides along at least one section of a longitudinal extension of the patient couch 17. The side elements 31 are designed to prevent a contact of an anatomical region of the patient 15 with a wall of the patient receiving zone 14 or patient tunnel.

The patient support apparatus 16, according to the aspects of the disclosure, additionally comprises two joints 34 that secure the side elements 31 on the patient support apparatus 16 and allow the side elements 31 to rotate about axes that are defined by the joints 34. The axes defined by the joints 34 may be aligned substantially parallel to a longitudinal extension of the patient couch 17 or parallel to the Z-axis of the patient support apparatus 16.

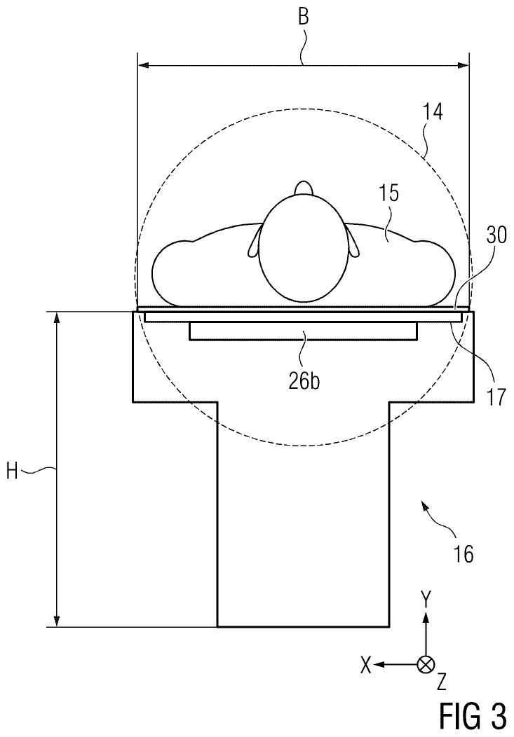

FIG. 3 shows an aspect of the patient support apparatus 16 according to the aspects of the disclosure, which dispenses with a provision of side elements 31. However, the side elements 31 can also be reversibly mountable (e.g., pluggable or clampable) on the patient couch 17, the patient support apparatus 16, and/or the cushioning element 30.

In the example shown in FIG. 3 also, the width B of the cushioning element 30 substantially corresponds to a width of the patient receiving zone 14 at a receiving height H for the patient couch 17. It is conceivable that a diameter of the patient receiving zone 14 exceeds the width B of the cushioning element 30.

The cushioning element 30 shown in FIGS. 2 and 3 makes maximum use of the available width of the patient receiving zone 14 at the receiving height H for the patient couch 17. The cushioning element 30 preferably covers an entire surface of the patient couch 17 and of the patient support apparatus 16 facing in the direction of the patient 15 positioned in an appropriate manner for examination on the patient couch 17. As a result, all of the patient 15 positioned in an appropriate manner for examination on the patient couch 17 rests on the cushioning element 30, and additional operating steps of the underpadding can advantageously be avoided.

It is conceivable that the patient support apparatus 16 comprises a spine coil 26b, which is integrated into the patient couch 17 or into the patient support apparatus 16 in the immediate vicinity of the patient couch 17. A molding to conform to the body of the patient 15 is made possible thanks to the elastic material of the cushioning element 30, as a result of which an average distance between the patient 15 and the spine coil 26b can be reduced. Typically, the signal-to-noise ratio of the spine coil 26b is reduced by about 10% per cm distance from the patient 15. As a result of the patient 15 “sinking” into the cushioning element 30 according to the aspects of the disclosure, the highest possible signal-to-noise ratio can therefore be ensured.

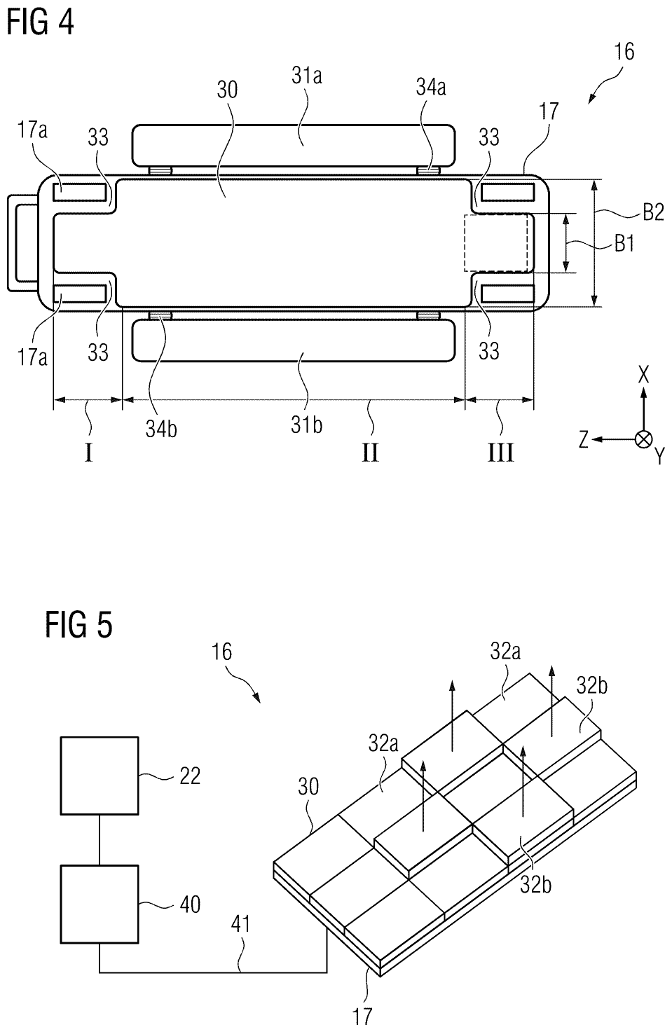

FIG. 4 shows a plan view onto an aspect of a patient support apparatus 16 according to the aspects of the disclosure. In the example shown, the patient support apparatus 16 has at least two joints 34a and 34b, which enable the side elements 31a and 31b to be rotated or tilted about axes defined by the joints 34a and 34b. The side elements 31a and 31b can therefore be pivoted into an open position, which facilitates access for the patient to the patient couch 17. When the patient 15 is positioned in an appropriate manner for examination on the patient couch 17, the side elements 31a can be pivoted into an examination position in which the patient 15 is flanked by the two side elements 31a and 31b from two opposite sides along at least a part of the longitudinal extension of the patient couch 17 (see FIG. 2).

In further aspects, the side elements 31a and 31b can be connectable to the patient couch 17, the cushioning element 30, and/or the patient support apparatus 16 by means of a mechanical connection, e.g., a snap-fit connection, a screwed connection, a clamped connection, and/or a latching mechanism. It is furthermore conceivable that the side elements 31a and 31b are designed as lowerable into the patient couch 17 and/or the patient support apparatus 16.

In the example shown in FIG. 4, the cushioning element 30 is subdivided into three sections I, II, and III. The cushioning element 30 has a plurality of recesses 33. The recesses 33 are designed to accommodate a component of the imaging apparatus 10. In the present example, the recesses 33 leave a free space for a rail or a fastening mechanism 17a for a local coil 26 (not shown). Owing to the recesses 33, sections I and III have a reduced width B1 in the X direction of the patient support apparatus 16 compared to section II. The width B2 of section II, on the other hand, substantially corresponds to the width of the patient receiving zone 14 at a receiving height H for the patient couch 17 (see also FIGS. 2 and 3).

The patient support apparatus 16 according to FIG. 4 has a section with a reduced width B both at a head end and at a foot end. However, it is also conceivable that one or more recesses 33 for housing a component of the imaging apparatus or a section with reduced width are present only on one side of the patient support apparatus 16.

FIG. 5 shows a further aspect of a patient support apparatus according to the aspects of the disclosure. In the example shown, the cushioning element 30 has a plurality of elastic elements 32, which are designed to support different anatomical regions of a patient 15 when the patient 15 is positioned in an appropriate manner for examination on the patient couch 17. In the present example, elastic elements 32 can be filled with a fluid by means of a fluid connection 41 in order to adjust a dimension and/or an elasticity of the elastic elements 32. For this purpose, the fluid connection 41 is connected to a conveyor device 40, e.g., a pump or a compressor, which is designed to transport a fluid from a reservoir (not shown) into the elastic elements 32 and vice versa. The conveyor device 40 is connected to the control unit 22 of the imaging apparatus by means of a signal connection. Preferably, the control unit 22 is designed to detect or identify an imaging examination that is to be performed on a patient 15 and to direct the conveyor device 40 to fill or empty one or more elastic elements 32 with the fluid in accordance with the imaging examination. In the example shown In FIG. 5, the elastic elements 32b are filled with a larger volume of a pneumatic fluid, such as e.g., air or an inert gas, than the elastic elements 32a. The dimensions and elastic properties of the elastic elements 32b are therefore different from the dimensions and elastic properties of the elastic elements 32a.

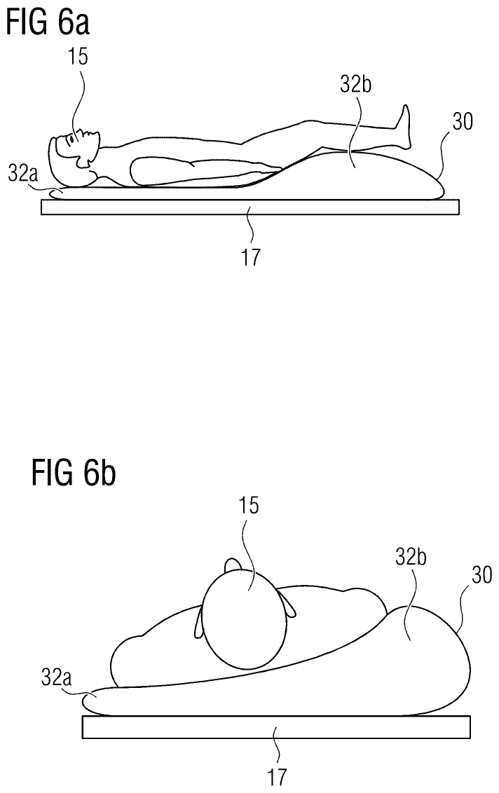

FIGS. 6a and 6b show examples in which a patient 15 is supported by means of the elastic elements 32b in assuming a predetermined position and/or posture for an imaging examination and/or to maintain said position and/or posture during the imaging examination.

FIG. 6a shows a leg of the patient 15 being held by means of the elastic element 32b at a predetermined distance from sides of the patient couch 17 facing toward the cushioning element 30 such that a section of the leg can be positioned in an imaging volume of the imaging apparatus 10.

In FIG. 6b, one or more elastic elements 32b on one side of the cushioning element 30 along a longitudinal extension of the patient couch 17 are inflated or filled with a larger volume of a fluid such that a right half of the body of the patient 15 is arranged at a greater distance from the patient couch 17 than the left half of the body of the patient 15.

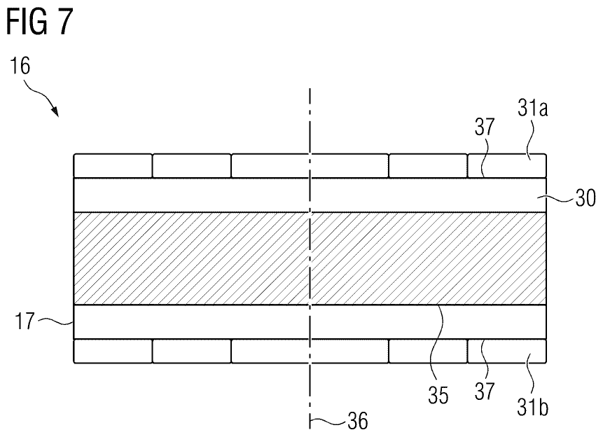

FIG. 7 shows a further plan view onto a patient support apparatus 16 according to the aspects of the disclosure. In the example shown, the cushioning element 30 has a marker 35 which represents a course of a projection of an imaging volume provided by the magnetic resonance device 10 onto the cushioning element 30 when the patient couch 17 is transported into the patient receiving zone 14 (see FIG. 1) by means of the patient support apparatus 16. The marker supports a user of the magnetic resonance device 10 or a member of the medical staff in positioning a patient 15 properly relative to the imaging volume 52 of the magnetic resonance device 10 on the patient couch 17. In particular, the marker 35 can support a member of the medical staff in positioning extremities or other anatomical regions of the patient 15 in a correct position relative to the imaging volume 52 of the imaging apparatus 1.

In the example shown in FIG. 7, the cushioning element 30 is designed as foldable along the fold line 36 in order to facilitate a removal of the cushioning element 30 from the patient couch 17 and/or a fitting of the cushioning element 30 onto the patient couch 17. The cushioning element 30 may have further fold lines 36 in order to simplify a removal or fitting process and/or a transportation of the cushioning element 30.

In the example shown, the optional side elements 31a and 31b are materially connected to the cushioning element 30 by means of the folding elements 37. However, it is also conceivable that the side elements 31a and 31b are secured to the cushioning element 30, the patient couch 17 and/or the patient support apparatus 16 by means of a reversible mechanical connection, in particular a clamped connection or a snap-fit connection, and can be removed from the patient support apparatus 16.

Although the aspects of the disclosure have been illustrated and described in more detail on the basis of the preferred exemplary aspects, the invention is nonetheless not limited by the disclosed examples and other variations can be derived herefrom by the person skilled in the art without leaving the scope of protection of the invention.

1. A patient support apparatus for an imaging apparatus, the patient support apparatus comprising:
a patient couch; and
a cushioning element arranged on a surface of the patient couch and forming a receiving surface for a patient,
wherein the patient support apparatus is designed to transport the patient on the cushioning element using the patient couch into a patient receiving zone of the imaging apparatus, and
wherein a width of the cushioning element along an X direction of the patient support apparatus substantially corresponds to a width of the patient receiving zone at a receiving height for the patient couch.

2. The patient support apparatus as claimed in claim 1, further comprising:
a side element which is arranged along a longitudinal edge of the patient couch and is designed to limit a patient positioned in an appropriate manner for examination on the patient couch on one side along at least a part of a longitudinal extension of the patient.

3. The patient support apparatus as claimed in claim 2, further comprising:
a joint which is designed to variably change a spatial arrangement of the side element relative to the patient couch.

4. The patient support apparatus as claimed in claim 3, further comprising:
an actuator which is designed to guide the side element variably using the joint along a movement trajectory defined by the joint.

5. The patient support apparatus as claimed in claim 2, wherein the side element has a flexible section which is designed to be adjusted in shape to conform to a surface of a patient positioned in an appropriate manner for examination on the patient couch.

6. The patient support apparatus as claimed in claim 2, further comprising:
a fixing element which is designed to secure the side element in a predetermined arrangement relative to the patient couch and/or to a patient positioned in an appropriate manner for examination on the patient couch.

7. The patient support apparatus as claimed in claim 1, wherein the cushioning element has a recess which is designed to accommodate a component of the imaging apparatus.

8. The patient support apparatus as claimed in claim 1, wherein the width of the cushioning element along an entire longitudinal extension of the patient couch substantially coincides with the width of the patient receiving zone at the receiving height for the patient couch.

9. The patient support apparatus as claimed in claim 1, wherein the cushioning element has a plurality of elastic elements which are designed to support selected anatomical regions of a patient when the patient is positioned in an appropriate manner for examination on the patient couch.

10. The patient support apparatus as claimed in claim 9, wherein a first elastic element and a second elastic element of the plurality of elastic elements project at different heights from a side of the patient couch facing toward the cushioning element when the cushioning element is arranged in an appropriate manner for use on the patient couch.

11. The patient support apparatus as claimed in claim 9, further comprising a shaping device which is designed to adjust an elasticity and/or a dimension of an elastic element of the plurality of elastic elements in accordance with an imaging examination.

12. The patient support apparatus as claimed in claim 11, wherein the plurality of elastic elements have at least one fillable element, and wherein a shaping unit is designed to fill the at least one fillable element with a fluid in order to change the elasticity and/or the dimension of the at least one element.

13. An imaging apparatus comprising:
an image acquisition region; and
a patient support apparatus as claimed in claim 1,
wherein the imaging apparatus is designed to acquire diagnostic image data of an object positioned in the image acquisition region.

14. The imaging apparatus as claimed in claim 13,
wherein the cushioning element has a plurality of elastic elements which are designed to support selected anatomical regions of a patient when the patient is positioned in an appropriate manner for examination on the patient couch, and
wherein the patient support apparatus further comprises:
a shaping device which is designed to adjust an elasticity and/or a dimension of an elastic element of the plurality of elastic elements in accordance with an imaging examination; and
a control unit designed to direct the shaping device to adjust an elasticity and/or a dimension of an elastic element of the plurality of elastic elements in accordance with an imaging examination.

15. The imaging apparatus as claimed in claim 13, wherein the imaging apparatus is designed as a magnetic resonance device, and wherein the cushioning element has a marker which represents a course of a projection of an imaging volume provided by the magnetic resonance device onto the cushioning element when the patient couch is transported into the patient receiving zone by means of the patient support apparatus.
